(19) United States
(12) Patent Application Publication
(10) Pub. No.: US 2007/0038292 A1
(43) Pub. Date: Feb. 15, 2007

(54) Bio-absorbable stent

(76) Inventor: Moise Danielpour, Los Angeles, CA (US)

Correspondence Address: JONES DAY, 555 SOUTH FLOWER STREET FIFTIETH FLOOR, LOS ANGELES, CA, 90071, US

(21) Appl. No.: 11/200,763

(22) Filed: Aug. 9, 2005

(51) Int. Cl. A61F 2/94; A61F 2/06

(52) U.S. Cl. 623/1.42; 623/23.67; 623/1.39

(57) ABSTRACT

The present invention is a novel implantable medical device comprised of a tubular double lumen, radially compressible, axially flexible and expandable, bio-absorbable “bladder”-type stent.

### FIELD OF THE INVENTION

The present invention relates generally to implantable, bio-absorbable medical prostheses. In particular, the present invention relates to bio-absorbable, inflatable stents.

### BACKGROUND OF THE INVENTION

Intraluminal stents are commonly used for the treatment of various vascular and other luminal stenotic conditions, such as arteriosclerosis, often as coronary artery implants, carotid stents and stents across wide-based aneurysmal dilations of aneurysm sacks. A stent can be implanted at the site of a vessel stricture or stenosis using a conventional balloon catheter delivery system or as a self-dilating coil device introduced in its radially compressed longitudinally elongated form and expanding upon deployment out of the introduction. Such stents also may be deployed in a body passageway to treat strictures or prevent luminal occlusion. These stents typically consist of a cylindrical network of very small metal wires or bioabsorbable polymeric compounds intertwined into helices, etc. Such stent structures and implantation techniques are well known.

Great efforts have been expended to modify metallic stents in order to eliminate stent-induced and/or inflammation-induced restenosis, and to effectively deliver therapeutic agents to lesion sites. Some advances in drug-coated metal stents have been made. However, metallic stents still present many potential and now well demonstrated vessel injury problems. Furthermore the delivery of medicine to a lesion site by local or systemic means is unsatisfactory with current technology.

Several patents have been filed in recent years attempting to utilize readily available and well described bioabsorbable materials to construct a temporary, biocompatible, bioabsorbable stent capable of delivering local drug therapy with adequate tensile strength to dilate and keep the vessel lumen patent. The implantation of these stents will preferably cause a generally reduced amount of acute and chronic trauma to the luminal wall. A stent that applies a gentle radial force against the wall and that is compliant and flexible with lumen movements is preferred for use in diseased, weakened, or brittle lumens. Such a stent will also optimally be able to withstand radially occlusive pressure from tumors, atherosclerotic plaque, and remodeling/scarring. To do all this in face of the present high risk of restenosis, the stent must induce very little or no inflammatory reaction. Current technology falls significantly short of these goals and potentially poses significant biological risks. The present invention aims to address all these shortcomings in a biologically viable stent.

### SUMMARY OF THE INVENTION

The present invention provides a novel bioabsorbable stent. In a preferred embodiment of the invention, the stent comprises a first lumen that is sealed by the stent's outer and inner walls. The first stent lumen is designed to be inflated in situ with a composition of choice. The second lumen, which is open-ended, extends longitudinally through the first, sealed lumen from which it is separated by the inner stent wall. The outer stent wall is designed to rest against a vessel wall and exert radial pressure against a vessel wall upon inflation of the first, sealed lumen. The stent itself is pliable but becomes rigid when filled with a composition of choice, preferably a liquid or a gel, and is able to exert radial force against the walls of a blood vessel, duct, or other lumen within the body to which it is deployed. In preferred embodiments, the composition of choice is a therapeutic composition and may be selected from antisclerotic, anticoagulant, and chemoattractant agents.

The stent can be inflated by filling the first lumen with a composition of choice, thereby increasing the diameter of both the stent and said second lumen until the stent is fully extended and the second lumen allows the unimpeded passage of a bodily fluid, such as blood, lymph, urine, bile, tear fluid etc. or other material. In preferred embodiments, the stent wall is made of a polymer, preferably a bioabsorbable polymer with the appropriate tensile strength for the particular application.

In preferred embodiments, said first, fillable lumen may comprise one or more separated sublumens or subcompartments which may be filled with different compositions of choice. In preferred embodiments, the first lumen comprises two or more sublumens located concentrically, i.e. one sublumen faces the external surface of the stent, while another faces the interior lumen of the stent, whereas additional sublumens can be located in-between these two sublumens. This arrangement allows for the selective release of one drug toward the vessel wall against which the stent rests and the selective release of another drug toward the vessel lumen. Alternatively, the one or more sublumens may be arranged in alternating striation-type pattern, or any other pattern suitable for the particular purpose intended. In preferred embodiments, the sublumens are connected to individual separate inflow sources.

The stent wall may further comprise pores of predetermined size to allow for the controlled release of a composition of choice from the first stent lumen. Pores of one or more sizes may be positioned in the inner stent wall facing the interior vessel lumen, whereas pores of another size may be positioned in the outer stent wall resting against the vessel wall in which the stent is employed. The outer and/or inner stent walls may additionally be coated with a composition of choice. Alternatively, the polymers of the stent walls may contain a composition of choice, including anticoagulants, chemoattractants, and antisclerotics, to name a few.

In preferred embodiments of the invention, the stent may further comprise a means for reversibly connecting to a fluid pressure source, such as a catheter. Connecting the stent to a fluid pressure source allows the stent to be reloaded with the same or a different composition of choice at various points in time. In preferred embodiments, said means for reversibly connecting to a fluid pressure source may be located in a readily accessible area. In yet other preferred embodiments, the stent may further comprise a pressure sensor.

### BRIEF DESCRIPTION OF THE DRAWINGS

FIG. 1 represents a cross-section of the stent of the present invention showing the inner (10) and outer (9) stent walls and the fillable lumen (1) located therebetween.

FIG. 2 shows another embodiment of the stent of the present invention with concentrically arranged fillable sublumens (4).

FIG. 3 shows an alternative embodiment of the stent with two separated fillable sublumens arranged in a striation-type of pattern, each fillable sublumen having its own fluid inflow source (6).

FIG. 4 shows the outer wall of the stent with apertures (7) that are of a different size than the apertures (7) located on the interior wall of the stent.

FIG. 5A shows the fillable lumen molded into a mesh-type configuration leaving.

FIG. 5B shows the fillable lumen molded into a helical configuration.

FIG. 6A shows an inflatable stent with pre-filled pockets (8) and the fillable lumen with an inflow source (6).

FIG. 6B shows the pocketed stent in cross-section.

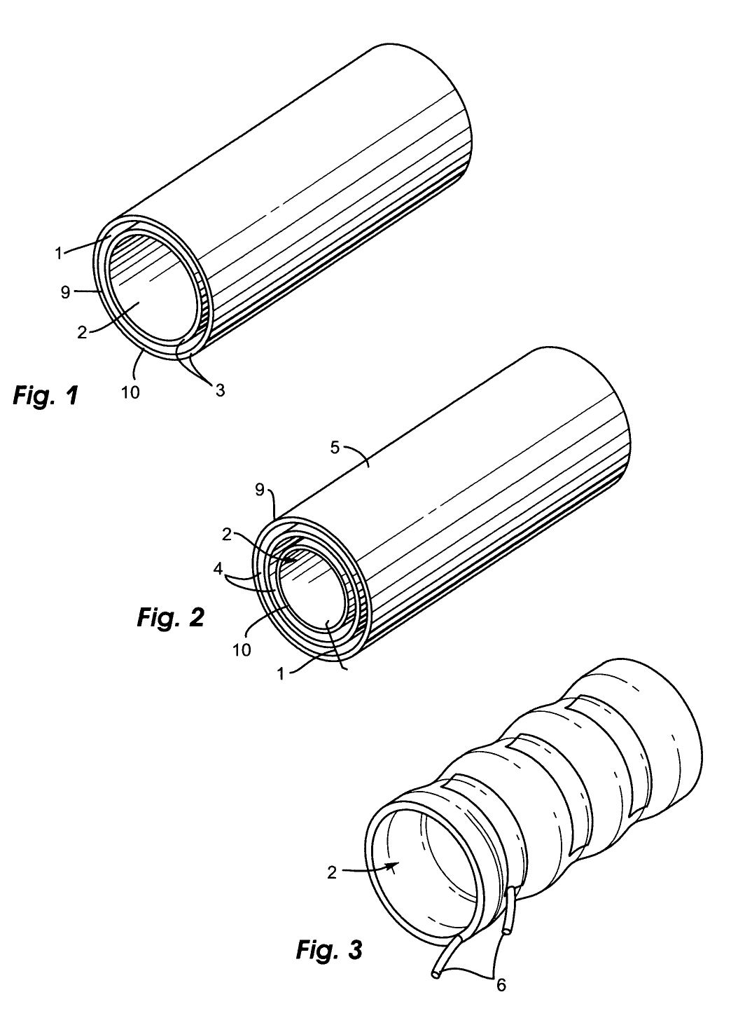

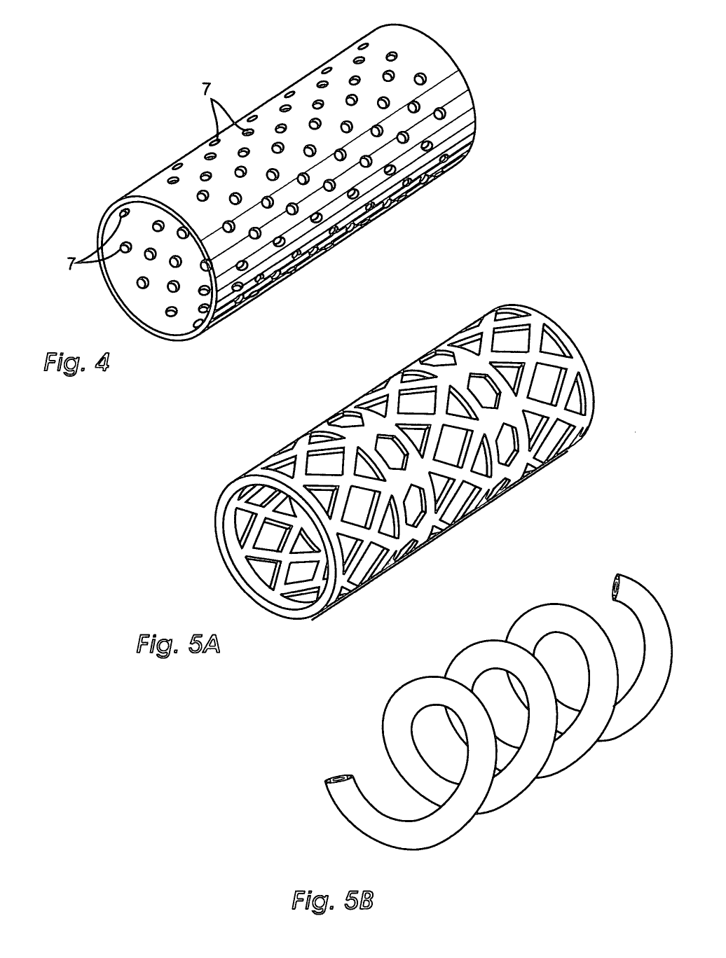

### DETAILED DESCRIPTION OF THE INVENTION

The present invention provides a novel bioabsorbable stent that is expandable by inflation with a composition of choice. In a preferred embodiment of the invention the stent comprises a first (1) and second (2) lumen, as well as an outer (9) and inner (10) stent wall. The first stent lumen (1) is sealed by the stent's outer and inner walls (3) and designed to be inflated in situ with a composition of choice. The second lumen (2) is open-ended and extends longitudinally through the first, sealed lumen from which it is separated by the inner stent wall (10). The stent itself is pliable due to the tensile strength of the polymeric material from which its walls are constructed, but becomes rigid when filled with a composition of choice, preferably a liquid or a gel. Thus, upon inflation, the stent of the present invention is able to exert radial force against the walls of a blood vessel, duct, or other lumen within the body to which it is deployed and able to resist compression. A valve-like mechanism may be employed to prevent deflation of the stent after deployment. Such mechanisms, including those with self-sealing properties, are well known in the art. Alternatively, mechanisms which detach with heating of a platinum electrode wire may be used to seal the inflated stent. By inflation or inflatable is meant the introduction of any composition of matter into the first stent lumen, which can include fluid, gel-like, liquid, gaseous, or solid-phase compositions (i.e. for example lyophilized material or nanoparticles), as well as combinations of such compositions. While reference may be made throughout this specification to such terms as inflow source and fluid pressure source, it should be understood that this is for purposes of linguistic simplicity only, and should not be understood as limiting the present invention to inflation by fluids.

In preferred embodiments, the composition of choice is a therapeutic composition and may perform a variety of functions including, but not limited to, anti-clotting or anti-platelet function; preventing microbial and/or smooth muscle cell growth on the inner surface wall of the vessel. The composition may also aid in visualizing the stent by imaging techniques, thus it may contain a radioopaque or contrast agent. Compositions suitable for the purposes of the present invention include but are not limited to drugs that inhibit or control the formation of thrombi or thrombolytics such as heparin or heparin fragments, prostacyclin, aspirin, coumadin, tissue plasminogen activator (TPA), urokinase, hirudin, and streptokinase, antiproliferatives (methotrexate, cisplatin, fluorouracil, adriamycin, and the like) antioxidants (ascorbic acid, carotene, B, vitamin E, and the like), antimetabolites, antibiotics, and radioactive agents for the delivery of radiation, thromboxane inhibitors, non-steroidal and steroidal anti-inflammatory drugs, beta- and calcium channel blockers, genetic materials including DNA and RNA fragments, including siRNA, complete expression genes, carbohydrates, and proteins including but not limited to antibodies (monoclonal and polyclonal) lymphokines and growth factors, prostaglandins, and leukotrienes. Generally, the composition of choice may be selected from antisclerotic, anticoagulant, antimicrobial, chemoattractant, radioopaque, or radioactive agents, or a combination thereof.

The stent can be inflated by filling the first lumen (1) with the composition or compositions of choice, thereby increasing the diameter of the second lumen (2) until the stent is fully extended and the second lumen (2) allows the unimpeded passage of a bodily fluid or other material, including blood, lymph, urine, bile, tear fluid, air, etc. In preferred embodiments of the invention, inflation of the stent takes place in situ, i.e. after the stent is deployed to its desired location. This allows for the deployment of the stent in a substantially compressed form and permits stent placement by minimally invasive techniques, such as catheter-, trocar-, or cannulation-based techniques. Mechanisms which detach with heating of a platinum electrode wire may be used to seal the inflated stent. Other mechanisms, including valve-like mechanisms made of bioabsorbable polymer may also be used for the purposes of the present invention. The stent of the present invention may optionally be deployed and placed with the aid of a balloon.

In preferred embodiments, the stent walls (3) are made of a polymer, preferably a bioabsorbable polymer with the appropriate tensile strength for the particular application. Examples of polymers suitable for the purposes of the present invention include biodegradable polymeric compounds, including polymers of lactic acid, poly(alpha-hydroxy acid) such as poly-L-lactide (PLLA), poly-D-lactide (PDLA), polyglycolide (PGA), polydioxanone, polyglycolic acids, polycaprolactone, polygluconate, polylactic acid-polyethylene oxide copolymers, modified cellulose, collagen, poly(hydroxybutyrate), polyanhydride, polyphosphoester, poly(amino acids), tyrosine-derived polycarbonates, poly-lactic-co-glycolide (PLGA) or related copolymers, as well as blends of the foregoing polymers, or their respective monomers, dimers, or oligomers, each of which have a characteristic degradation rate in the body. For example, PGA and polydioxanone are relatively fast-bioabsorbing materials (weeks to months) and PLA and polycaprolactone are a relatively slow-bioabsorbing material (months to years). All of these materials are readily available and well known to a person of skill in the art.

The stent wall may further comprise pores or apertures (7) of predetermined size to allow for the controlled release of a composition of choice from the first stent lumen. Pores of one or more sizes may be positioned in the inner stent wall facing the interior vessel lumen, whereas pores of another size may be positioned in the outer stent wall resting against the vessel wall in which the stent is employed. One advantage of the design of the present invention is the ability to deliver much larger quantities of therapeutic compositions to locations of choice, as the fillable lumen (1) of the stent is able to accommodate a significant amount of material as compared to the more limited ability of a stent coat to accommodate therapeutic agents. The amount of drug or therapeutic composition that can be delivered, as well as the time over which it is delivered, are thus vastly increased by stent of the present invention.

The fillable lumen (1) of the stent may also be inflated with drugs that can help dissolve an atherosclerotic plaque, act as anticoagulants to prevent distal emboli or chemoattractants to promote infiltration/recruitment of stem cells to site of injury. Undifferentiated stems cells have well demonstrated anti-inflammatory properties that may be important in later stages of healing after plaque resorption, to prevent restenosis. Therefore the unique design of this stent allows the deployment of different drugs/agents at different time points from deployment.

In preferred embodiments, the first, fillable lumen (1) may comprise one or more separated sublumens or subcompartments (4) which may be filled with different compositions of choice. In preferred embodiments, the first lumen (1) comprises two or more sublumens (4) located concentrically, i.e. one sublumen faces the external surface of the stent, while another faces the interior lumen of the stent, whereas additional sublumens can be located in-between these two sublumens, separated by stent walls (3). This arrangement allows for the selective release of one drug toward the vessel wall against which the stent rests and the selective release of another drug toward the vessel lumen. Alternatively, the one or more sublumens may be arranged in alternating striation-type pattern (FIG. 3), or any other pattern suitable for the particular purpose intended. In preferred embodiments, the sublumens are connected to their own separate inflow sources (6).

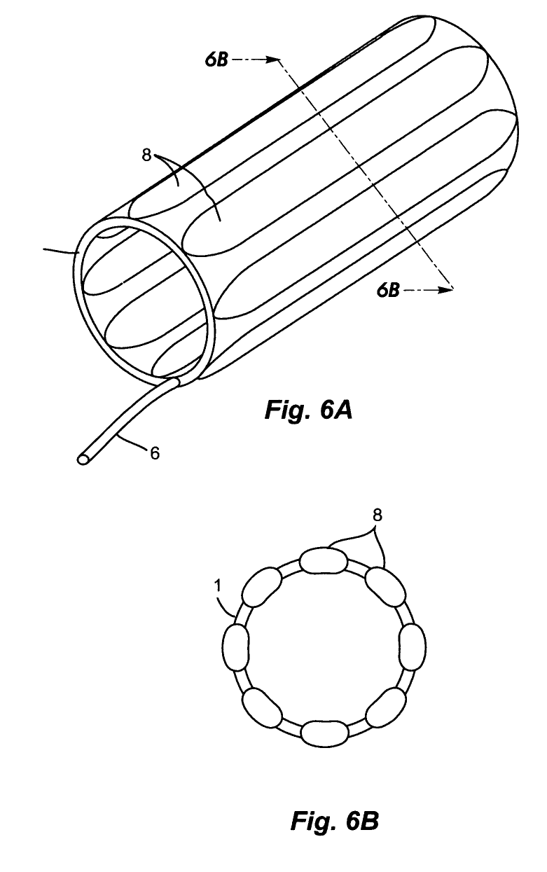

The walls of the stent (3) of the present invention can be molded to form either an uninterrupted double lumen or to form a mesh (FIG. 5A) or helical configuration (FIG. 5B). The helical or mesh configuration can be used if there are tributary vessels that are desired to be kept open or the stent is being used as an intraluminal support for coil material being inserted into a wide neck aneurysmal dilatation. The coil material can be delivered through the vessel lumen, across the intermittent gaps in the stent scaffolding and into the aneurysmal sac. In such a configuration, when used for treatment of an aneurysm, the stent scaffolding acts as a brace for decreasing the risk of the implanted coil backing out into the vessel lumen. The stent in the interrupted mesh or helical configuration may decrease any disruption of the intravascular laminar blood flow patterns, any disruption of which may theoretically increase the risk of increased stress on vessel walls at distal branch point or adjacent vessel wall. The stent may also decrease the risk of formation of abnormal eddies that increase the risk of coagulation and distal thrombotic emboli.

In a further embodiment of the present invention, the stent walls (3), both outer (9) and inner (10), may additionally be coated with a composition of choice. Alternatively, or additionally, the composition of choice may be embedded in the polymeric stent wall or covalently bound to it by processes well known in the art. Such compositions of choice may include anticoagulants, antimicrobials, chemoattractants, chemotherapeutics, antisclerotics, i.e. angiopeptin, methotrexate, heparin, as well as drugs that positively affect healing at the site where the stent is deployed, either incorporated into the polymer forming the stent, or incorporated into the coating, or both. Other suitable drugs may include antithrombotics (such as anticoagulants), antimitogens, antimitotoxins, antisense oligonucleotides, gene therapy vehicles, nitric oxide, and growth factors and inhibitors. Known direct thrombin inhibitors include hirudin, hirugen, hirulog, PPACK (D-phenylalanyl-L-propyl-L-arginine chloromethyl ketone), argatreban, and D-FPRCH₂ Cl (D-phenylalanyl-L-propyl-L-arginyl chloromethyl ketone); indirect thrombin inhibitors include heparin and warfarin. All of these compositions preferably are incorporated in quantities that permit desirable timed release as the stent and/or coating biodegrades.

A stent prepared according to the present invention preferably also incorporates surface coatings or thin films designed to reduce the risk of thrombosis and to deliver bioactive agents. These compositions can be blended or copolymerized with the biodegradable polymers of the stent walls (3). The outer (9) and inner (10) walls of the stent may also incorporate different compositions and combinations thereof, depending on the biological function desired. Bioactive materials such as fibronectin, laminin, elastin, collagen, and intergrins may be chosen for coating of or incorporation into the stent walls. For example, it may be desirable to coat the outer wall (9) (adjoining the vessel wall), but not the inner wall (10) (facing the vessel lumen) of the stent with fibronectin, because fibronectin is known to promote adherence of the stent to the tissue of the vessel or duct. The stent walls (3) may also be coated with different drugs that can not only act as anticoagulant, prevent adherence of white cells, but alternatively with chemoattractive compounds used to attract bone marrow derived stem cells to the site of vessel injury, dissection, atherosclerosis or vessel wall weakness.

In preferred embodiments of the invention, the stent may further comprise a means for reversibly connecting to a fluid pressure source, such as a catheter. Connecting the stent to a fluid pressure source allows the stent to be reloaded with the same or a different composition of choice at various points in time, further increasing the capacity of the stent to deliver therapeutic compositions and prolonging the exposure period of a body tissue to the therapeutic compositions. Any mechanism that allows the reversible re-connection between the fillable stent lumen (1) and a fluid pressure source is suitable for the purposes of the instant invention. Thus, any snap-on, screw-on, slide-on or other mechanism is contemplated for use herein. It should be noted that the present invention also allows for the simple deflation and removal of the stent, if indicated, by reversing the flow of fluid and directing it toward the source of fluid pressure.

In yet another preferred embodiment of the present invention, the bioabsorbable, inflatable stent may comprise pre-filled pockets (8) as shown in FIGS. 6A and 6B. These pockets are separated from the first stent lumen by the stent walls and (8) may be prefilled with any of the drugs of choice mentioned herein, or they may be prefilled with contrast. After the stent is placed in position, the fillable lumen (1) may be inflated via the inflow source (6) with contrast or any composition of choice to reach its fully expanded diameter at which it will exert spring action against the vessel walls. In a preferred embodiment, the stent may further comprise a pressure sensor. Said pressure sensor may be located near the means for reversibly connecting the stent to the source of fluid pressure, such as in a physically accessible location.

Thus, the present invention provides an inflatable bio-absorbable prosthetic device of biodegradable polymer which can be expanded or dilated by filling a first lumen with a composition or drug of interest to the desired pressure creating a thin walled biodegradable stent with improved radial and tensile strength and ability to deliver larger quantities of drug, both locally and distally, without compromising the ability of the stent to keep the vessel lumen patent.

1. A bioabsorbable stent comprising:
a) a first, sealed lumen having an outer and inner stent wall;
b) a second, open-ended lumen extending longitudinally through said first lumen and separated from said first lumen by said inner stent wall;
c) wherein said first lumen is inflatable.

2. The stent of claim 1, wherein said first lumen provides structural rigidity when inflated.

3. The stent of claim 1, wherein said first lumen is inflated with a therapeutic composition.

4. The stent of claim 3, wherein said therapeutic composition is selected from the group consisting of antisclerotic, anticoagulant, radioactive, and chemoattractant agents.

5. The stent of claim 1, wherein said first lumen is inflated with a contrast agent.

6. The stent of claim 3, wherein inflating said first lumen with a composition of choice enlarges the diameter of said second lumen.

7. The stent of claim 6, wherein said second lumen allows the passage of a bodily fluid or other material.

8. The stent of claim 1, wherein said outer and inner stent walls are made of a polymer.

9. The stent of claim 8, wherein said polymer is bioabsorbable.

10. The stent of claim 8, wherein said polymer provides tensile strength.

11. The stent of claim 8, wherein said polymer is selected from the group consisting of poly-L-lactide (PLLA), poly-D-lactide (PDLA), polyglycolide (PGA), poly-lactic-co-glycolide (PLGA), polydioxanone, polyglycolic acids, polycaprolactone, polygluconate, polylactic acid-polyethylene oxide copolymers, modified cellulose, collagen, poly(hydroxybutyrate), polyanhydride, polyphosphoester, poly(amino acids), and tyrosine-derived polycarbonates.

12. The stent of claim 1, wherein said outer stent wall comprises pores of predetermined sizes.

13. The stent of claim 1, wherein said inner stent wall comprises pores of predetermined sizes.

14. A bioabsorbable stent comprising:
a) a first, sealed, inflatable lumen having an outer and inner stent wall comprising pores of predetermined sizes;
b) a second, open-ended lumen extending longitudinally through said first lumen and separated from said first lumen by said inner stent wall;
c) wherein said pores allow the controlled release of a therapeutic composition from said first stent lumen.

15. The stent of claim 1, wherein said outer and/or inner stent walls are coated with a composition of choice.

16. The stent of claim 15, wherein said composition of choice is selected from the group consisting of anticoagulants, chemoattractants, antisclerotics. and contrast agents.

17. The stent of claim 1, wherein said first lumen comprises means for reversibly connecting to a fluid pressure source.

18. The stent of claim 17, wherein connecting a fluid pressure source to said means allows the stent to be reloaded with the same or a different composition of choice.

19. The stent of claim 18, further comprising a pressure sensor.

20. The stent of claim 1, wherein said first lumen comprises one or more sublumens.

21. The stent of claim 1, wherein said stent is optionally placed with the aid of a balloon.

22. A bioabsorbable stent comprising:
d) a first, inflatable, sealed lumen having an outer and inner stent wall;
e) wherein said outer and inner stent walls are coated with an anticoagulant;
f) a second, open-ended lumen extending longitudinally through said first lumen and separated from said first lumen by said inner stent wall;
g) wherein said first lumen is inflated in situ with a contrast agent.
